(19) United States
(12) Patent Application Publication
(10) Pub. No.: US 2014/0370616 A1
(43) Pub. Date: Dec. 18, 2014

(54) LATERAL FLOW IMMUNOASSAY FOR DETECTING VITAMINS

(71) Applicant: NANOSPEED DIAGNOSTICS INC., Edmonton (CA)

(72) Inventors: Rajan Gupta, Edmonton (CA); Seema Gupta, Edmonton (CA)

(73) Assignee: NANOSPEED DIAGNOSTICS INC., Edmonton (CA)

(21) Appl. No.: 14/007,006

(22) PCT Filed: Mar. 23, 2012
(86) PCT No.: PCT/CA2012/000266

§ 371 Date: Sep. 4, 2014

Related U.S. Application Data
(60) Provisional application No. 61/467,668, filed on Mar. 25, 2011.

(51) Int. Cl. G01N 33/82

(52) U.S. Cl. CPC G01N 33/82; G01N 2800/02
USPC 436/501; 422/69

(57) ABSTRACT

The invention is directed to a method for detecting analytes, particularly vitamins, using a lateral flow immunoassay. The method may be used to detect vitamin D, particularly 25-hydroxy vitamin D₃ or 1,25-dihydroxy vitamin D₃. The method involves obtaining a fluid sample from a subject; applying the sample to a lateral flow test strip comprising a conjugate pad capable of releasing a labelled antibody against vitamin D, and a detection membrane comprising a first capture reagent specific to the antibody and immobilized on a test band; and detecting the presence or absence of vitamin D in the sample. The presence of a detectable signal in the test band is indicative of vitamin D deficiency.

### FIELD OF THE INVENTION

The present invention is directed to a method for detecting or quantifying analytes, particularly a vitamin, using a lateral flow immunoassay.

### BACKGROUND OF THE INVENTION

Vitamin D is a group of fat-soluble prohormones, of which the two major forms are vitamin D₂ (or ergocalciferol) and vitamin D₃ (or cholecalciferol). Vitamin D₃ is produced in skin exposed to sunlight, and is obtained through consumption of fish liver oils, salt water fish, mushrooms, fortified milk or other foods, and dietary supplements. Vitamin D₃ is biologically inert and must undergo two hydroxylations in the body for activation. First, the liver converts vitamin D₃ to 25-hydroxy vitamin D (25(OH)D or calcidiol), which is then converted by the kidneys to physiologically active 1,25-dihydroxy vitamin D (1,25(OH)₂D or calcitriol).

Vitamin D promotes bone formation; muscle contraction; nerve conduction; absorption of calcium and phosphate in the gut; calcium reabsorption by the tubules; inhibition of parathyroid hormone secretion; stimulation of insulin production; and immunogenic and antitumor activity.

Since vitamin D affects many organ systems including bone, the immune system, intestine, kidneys, parathyroid glands, and pancreas, hypovitaminosis D or vitamin D deficiency is a significant worldwide health concern. Vitamin D deficiency is widespread among the populations of the world, particularly in northern latitudes (for example, Canada and the United Kingdom) which experience reduced sunlight during winter. The deficiency can also result from inadequate nutritional intake of vitamin D, disorders which limit vitamin D absorption, conditions which impair the conversion of vitamin D into active metabolites, resistance to the effects of vitamin D, and use of certain medicines including phenytoin, phenobarbital and rifampin.

Vitamin D deficiency results in impaired bone mineralization, rickets in children, and osteomalacia and osteoporosis in adults. Although it was initially thought to be prevalent in northern latitudes, Vitamin D deficiency is equally common among tropical developing regions having equatorial climates. Vitamin D deficiency and nutritional rickets remains a growing concern. It ranks among the five most common diseases in children and is expected to be most common among infants with limited sunlight exposure, limited dietary supplements and mothers with poor vitamin D reserves. Vitamin D sources in early infancy comprise of trans-placental stores, breast milk and cutaneous production via sunlight. Although sunshine is available in most of the populated regions in world, reports suggest that a minimum of 25% of adolescents and more than 50% of undernourished communities in developed countries are vitamin D₃ deficient, leading to severe osteomalacia and osteoporosis in adults and rickets in infants. The situation is worse among women during pre-partum and post-partum periods due to indoor stays, reduced exposure to sunshine, poor dietary calcium intake and no vitamin supplements. This situation adversely affects the active trans-placental transport of calcium and vitamin supplements to the developing fetus, leading to fetal hypovitaminosis D, and neonatal/infantile rickets in infants.

Vitamin D deficiency may also be linked to an increased susceptibility to several chronic diseases such as high blood pressure, tuberculosis, cancer, periodontal disease, multiple sclerosis, chronic pain, depression, Parkinson's and Alzheimer's disease, schizophrenia, seasonal affective disorder, peripheral artery disease, and several autoimmune diseases including rheumatoid arthritis and type 1 diabetes. Treatments for vitamin D deficiency include, for example, supplemental vitamin D, correction of calcium and phosphate deficiencies, and dietary counseling.

There is a significant demand for vitamin D testing. A Canadian Health Measure Survey (Statistics Canada, McGill University) found that 60% of Canadians have inadequate levels of vitamin D, costing the healthcare system $14 billion cumulatively. In Ontario alone, the demand for vitamin D testing exceeded $60 million annually, jumping from 29,000 tests in 2004 to more than 700,000 in 2009 at a cost of $85/test.

Current diagnostic tests for patients exhibiting symptoms of vitamin D deficiency include X-rays to observe bone changes, and quantification of the levels of serum 25(OH)D, which reflect vitamin D status and correlate with symptoms of vitamin D deficiency better than levels of other vitamin D metabolites. However, considerable variability exists among current methods which are based upon for example, liquid chromatography-tandem mass spectrometry (Carter, 2009); competitive binding protein assays (Hollis, 2004); radio-immunoassays, chemiluminescent assays, solvent extraction and HPLC (Binkley et al., 2004; Snellman et al., 2010); specially designed recognition molecules for holo-DBP (Lawlor et al., 2010); and automated immunoassays (Wagner et al., 2009). Using such methods, it may take at least one to two weeks to obtain the test results. Further, current analytical instruments tend to be time-consuming and complex, requiring sophisticated experimental set-ups, specialized training, and considerable expense. The testing of vitamin D is an important tool for physicians to diagnose and treat vitamin D deficiency in patients.

### SUMMARY OF THE INVENTION

The present invention is directed to a methods and kits for detecting vitamin D using a lateral flow immunoassay.

In one aspect, the invention comprises a method of screening a fluid sample for a threshold quantity of vitamin D, comprising the steps of:
(a) obtaining a fluid sample from a subject;
(b) applying the sample to a lateral flow test strip comprising a conjugate pad comprising a known quantity of a labeled antibody against vitamin D;
(c) allowing an immunocomplex to form between the labeled antibody and any vitamin D in the sample;
(d) flowing the labeled antibody or immunocomplex through a test band comprising an immobilized first capture reagent capable of binding to:
   i. the labeled antibody but not the immunocomplex, or
   ii. the immunocomplex but not the labeled antibody; and
(e) determining whether or not the amount of vitamin D in the sample exceeds a threshold value by detecting or not detecting the labeled antibody in the test band.

In one embodiment, the labeled antibody but not the immunocomplex forms a visible signal in the test band. In an alternative embodiment, the immunocomplex but not the labeled antibody forms a visible signal in the test band.

In one embodiment, before step (b), the sample is treated to facilitate the release of vitamin D from a binding protein or a binding agent. In one embodiment, the sample is treated with urea, guanidine hydrochloride, an acidic solution, or an alkaline solution.

In one embodiment, the sample is a body fluid selected from urine, blood, plasma, serum, saliva, ocular fluid, spinal fluid, or perspiration. In one embodiment, the sample is a body fluid selected from serum or blood.

In one embodiment, the labeled antibody is anti-25-hydroxy vitamin D₃, or anti-25-hydroxy vitamin D₂, or anti-1,25-dihydroxy vitamin D₃. In one embodiment, the antibody is labeled with a label selected from a chromogen, a catalyst, a fluorescent compound, a chemiluminescent compound, a colloidal gold particle, a dye particle, or a latex particle tagged with a detector reagent. In one embodiment, the label comprises colloidal gold.

In one embodiment, the first capture reagent comprises 25-hydroxy vitamin D₃, 25-hydroxy vitamin D₂, or 1,25-dihydroxy vitamin D₃ conjugated to a carrier protein. The carrier protein aids in immobilizing the first capture reagent in the test band. In one embodiment, the carrier protein is selected from bovine serum albumin, keyhole limpet hemocyanin, thryoglobulin, or ovalbumin.

In one embodiment, the detection membrane further comprises a control band comprising a second capture reagent specific to an antibody or antigen and which is immobilized in the control band. In one embodiment, the second capture reagent comprises anti-mouse IgG antibody.

In one embodiment, the lateral flow test strip is housed within a cassette defining a sample aperture for introducing the sample and a test window for viewing a test result. In one embodiment, the cassette defines a buffer aperture for introducing dilution buffer.

In one embodiment, the amount of the sample may range from about 1 μL to about 100 μL. In one embodiment, the detectable signal is observed in about ten minutes or less.

In another aspect, the invention comprises a lateral flow immunoassay comprising a lateral flow test strip comprising a conjugate pad capable of releasing a labelled antibody against vitamin D, whereby the labeled antibody and any sample vitamin D forms an immunocomplex, and a detection membrane comprising an immobilized capture reagent capable of binding to i. the labeled antibody but not the immunocomplex, or ii. the immunocomplex but not the labeled antibody.

In yet another aspect, the invention may comprise a kit comprising the lateral flow test strip described above, a sample collector, and a diluent.

Additional aspects and advantages of the present invention will be apparent in view of the description, which follows. It should be understood, however, that the detailed description and the specific examples, while indicating preferred embodiments of the invention, are given by way of illustration only, since various changes and modifications will become apparent to those skilled in the art from this detailed description.

### BRIEF DESCRIPTION OF THE DRAWINGS

The invention will now be described by way of an exemplary embodiment with reference to the accompanying simplified, diagrammatic, not-to-scale drawings. In the drawings:

FIGS. 1A and 1B are top plan views of one embodiment of a test cassette of the present invention showing possible outcomes of the immunoassay through the test cassette window.

FIG. 1A shows a positive reaction having a stripe in only the control band. FIG. 1B shows a negative reaction having stripes in both the test and control bands.

FIG. 2 is a top plan view of one embodiment of a test cassette of the present invention including a buffer aperture for receiving dilution buffer.

FIG. 3 is a photograph showing the test strips removed from the test cassettes of FIGS. 1A and 1B and indicating possible outcomes of the immunoassay (positive reaction—lower panel; negative reaction—top panel).

FIG. 4 is a photograph showing test results of vitamin D deficient and sufficient blood samples.

### DETAILED DESCRIPTION OF PREFERRED EMBODIMENTS

The present invention is directed to a method for detecting vitamin D using a lateral flow immunoassay. When describing the present invention, all terms not defined herein have their common art-recognized meanings. To the extent that the following description is of a specific embodiment or a particular use of the invention, it is intended to be illustrative only, and not limiting of the claimed invention. The following description is intended to cover all alternatives, modifications and equivalents that are included in the spirit and scope of the invention, as defined in the appended claims.

Lateral flow immunoassays are simple tests for rapid detection of the presence or absence of a target analyte in a sample for home testing, point of care testing, or laboratory applications. Lateral flow test strips utilize a solid support through which a mobile phase (e.g., a liquid sample) can flow through by capillary action to a reaction matrix where a detectable signal, such as color changes or color differences on the test strip, may be generated to indicate the presence or absence of the target analyte. As used herein, the term “capillary action” or “capillarity” means the process by which a molecule is drawn across the lateral test strip due to such properties as surface tension and attraction between molecules.

In one embodiment, the present invention is directed to a lateral flow immunoassay for detecting vitamin D in a fluid sample. As used herein, the term “vitamin D” is meant to include all forms of vitamin D₂ and vitamin D₃. In one embodiment, the analyte is 25-hydroxy vitamin D₃, 25-hydroxy vitamin D₂, or 1,25-dihydroxy vitamin D₃. In one embodiment, the fluid sample is a human or animal body fluid such as serum or blood.

As used herein, the term “sample” means a fluid sample which may contain the vitamin. A sample may comprise a liquid body fluid (for example, urine, blood, plasma, serum, saliva, ocular fluid, spinal fluid, perspiration, and the like) from humans or animals.

The invention comprises a lateral flow immunoassay (10) for vitamin D. The immunoassay may be a competitive or non-competitive binding immunoassay, the principles of which are well known to those skilled in the art.

In one aspect, the invention comprises a method of screening a fluid sample for a threshold quantity of vitamin D, comprising the steps of:
(a) obtaining a fluid sample from a subject;
(b) applying the sample to a lateral flow test strip comprising a conjugate pad comprising a known quantity of a labeled antibody against vitamin D;
(c) allowing an immunocomplex to form between the labeled antibody and any vitamin D in the sample;
(d) flowing the labeled antibody or immunocomplex through a test band comprising an immobilized first capture reagent capable of binding to:
   i. the labeled antibody but not the immunocomplex, or
   ii. the immunocomplex but not the labeled antibody; and
(e) determining whether or not the amount of vitamin D in the sample exceeds a threshold value by detecting or not detecting the labeled antibody in the test band.

In one embodiment, the immunoassay comprises a competitive binding assay, where the labeled antibody but not the immunocomplex forms a visible signal in the test band. If a sufficient amount of vitamin D is present in the sample, there will remain none or only an insubstantial amount of unbound labeled antibody. Thus, if a sample contains sufficient vitamin D, the labeled antibody will not bind to the test antigen vitamin D, and will not be detectable in the test band. This result will be referred to herein as a positive result, indicating that it is positive for a sufficient amount of vitamin D in the sample.

In an alternative embodiment, the immunocomplex but not the labeled antibody forms a visible signal in the test band. Therefore, if a sufficient amount of vitamin D is present in the sample, a sufficient amount of immunocomplex will form and will be detectable in the test band. This result is also a positive result, indicating that it is positive for a sufficient amount of vitamin D in the sample.

In one embodiment, an immunoassay test strip (12) comprises a sample pad (14), a conjugate pad (16), a detection membrane (18), a wick (20), and a backing (22). In this assay, the conjugate pad comprises mobile labeled antibody while the detection membrane comprises an immobilized antigen. The labeled antibody will bind to the immobilized antigen, unless it is blocked by antigen present in the sample.

The sample pad (14) receives the sample and may be comprised of a fibrous material which absorbs the sample. The sample pad (14) may be formed of cotton, glass fiber, rayon, polyester, nylon, cellulose, spun polyethylene, or other suitable materials.

The sample is then drawn into the conjugate pad (16) which releases a labeled antibody into the sample. The conjugate pad (16) may be formed of polyesters, rayons or glass fibers. As used herein, the term “antibody” means a single antibody protein molecule or fragments thereof containing one or more variable antigen binding domain(s) and constant regions. Antibodies bind by means of specific binding sites to specific antigenic determinants or epitopes on antigens. The antibody is specific to an antigen (i.e., the analyte which may be present in the sample) and capable of binding to the antigen to form an antibody-antigen complex. As used herein, the term “antibody specific to” refers to an antibody which does not bind significantly to any sample components other than the desired component. In one embodiment, the antibody is an antibody specific to 25-hydroxy vitamin D₃, 25-hydroxy vitamin D₂, or 1,25-dihydroxy vitamin D₃. As used herein, the term “binding” means an interaction or complexation between an antibody and antigen, resulting in a sufficiently stable complex.

The antibody is conjugated to a detectable label which provides a means of visualizing or detecting the antibody-antigen complex, and may comprise a chromogen, catalyst, fluorescent compound, chemiluminescent compound, colloidal gold, a dye particle, a latex particle tagged with a detector reagent such as, for example, a colored or fluorescent dye, and the like. In one embodiment, the label comprises colloidal nanoparticulate gold. In one embodiment, the gold particles have a diameter size in the range of about 20-55 nm, preferably 35-45 nm. In one embodiment, the labeled antibody comprises an antibody specific to 25-hydroxy vitamin D₃, 25-hydroxy vitamin D₂, or 1,25-dihydroxy vitamin D₃.

After crossing the conjugate pad, the sample then passes into the detection membrane (18), which comprises a test band (26), and may also comprise a control band (24). The detection membrane may be comprised of nitrocellulose or a similar blotting material. The test and control bands (24, 26) each comprise a suitable capture reagent which has been immobilized in a particular area of the detection membrane (18) to “capture” or bind a specific molecule. As used herein, the term “immobilized” means the capture reagent is attached to or confined within the control band (24) or test band (26) such that lateral flow of fluids across the test strip (12) during the immunoassay (10) will not dislodge the capture reagent. In one embodiment, the control band (24) is positioned downstream of the test band (26).

In one embodiment, a first capture reagent immobilized in the test band (26) comprises 25-hydroxy vitamin D₃, 25-hydroxy vitamin D₂, or 1,25-dihydroxy vitamin D₃. Accordingly, any labeled antibody which is not part of an immunocomplex will bind to the first capture reagent. The first capture reagent is immobilized in the test band by conjugating it to a bulky protein. Suitable proteins have a size which is larger than the pore size of the detection membrane, and may include, but are not limited to, bovine serum albumin, keyhole limpet hemocyanin, thryoglobulin, and ovalbumin. In one embodiment, the protein is selected from bovine serum albumin or keyhole limpet hemocyanin. The bulky protein prevents migration of the first capture reagent through the detection membrane.

In an alternative embodiment, the immunoassay may comprise a sandwich assay in which the antigen/antibody complex, if present, also binds to an antibody specific to the vitamin which is fixed in the test band. Thus, the first capture reagent may comprise another antibody specific to the vitamin. As a result, the immunocomplex, but not unbound labeled antibody will bind to the test band and be detectable. In this case, a positive result will be indicated by the presence of a detectable signal in the test band.

The control band (24) comprises a capture reagent which will bind to the labeled antibody regardless of whether or not it has bound to the antigen vitamin D. For example, the control band capture reagent may comprise an immobilized antibody specific to the antibody portion of the antigen/antibody complex.

The wick (20) is downstream from the detection membrane, and serves to “pull” the fluids added to the test strip (12) for the duration of the immunoassay (10) by absorbing the fluids. Preferably, the wick (20) is of sufficient capacity and absorption ability to ensures that fluids do not backflow into the detection membrane (10), which may compromise the test results. The wick (20) is formed of a hydrophilic material, such as high-density cellulose, glass fibre/cellulose mix, cotton linter or other suitable materials.

The backing (22) serves as a physical support or base upon which the sample pad (14), the conjugate pad (16), the detection membrane (18), and the wick (20) are mounted. In one embodiment, the backing (22) is formed of a plastic material strip such as, for example, polystyrene. The test components (14, 16, 18, 20) may be mounted to the backing (22) by an adhesive. The components (14, 16, 18, 20) are mounted so as to abut each other, or overlap onto one another to maintain the flow of fluids across the test strip (12).

The test strip (12) and its components may be fabricated using techniques known to those skilled in the art. The conjugate pad (16) is pre-treated with labeled antibody by dispensing or dipping, followed by drying. The capture reagents at the control and test bands (24, 26) can be immobilized using several methods well known to those skilled in the art including, for example, direct adsorption and covalent attachment. Blocking of non-specific binding may be achieved by coating the surface of the detection membrane (18) with blocking buffers such as for example, bovine serum albumin, followed by drying. The sample pad (14) may also be pre-treated to filter out particulates, bind sample components which might interfere with the immunoassay (10), or disrupt the sample to release the target analyte. The components (14, 16, 18, 20, 22) are assembled into cards, with the sample pad (14), conjugate pad (16), detection membrane (18), and wick (20) being mounted onto the backing (22) using an appropriate adhesive. The cards are then cut into individual strips (12).

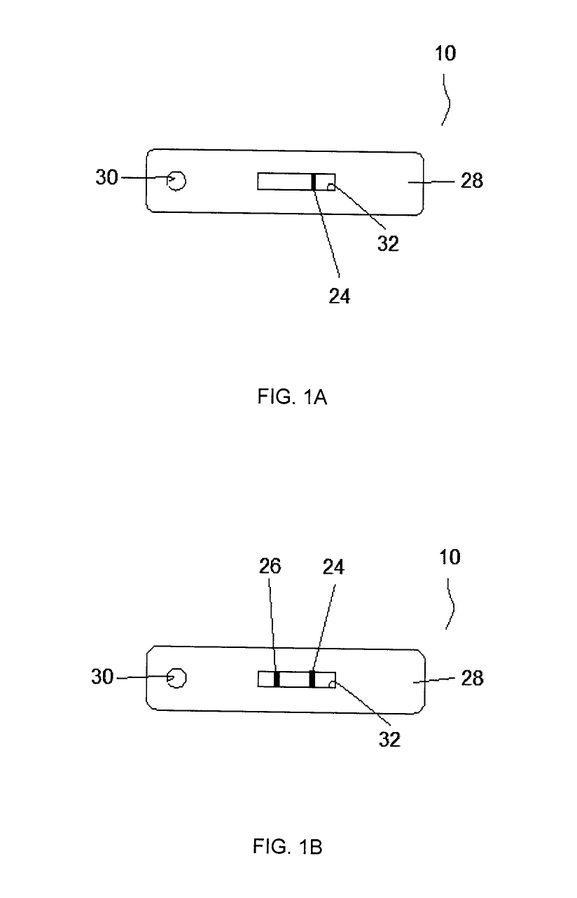

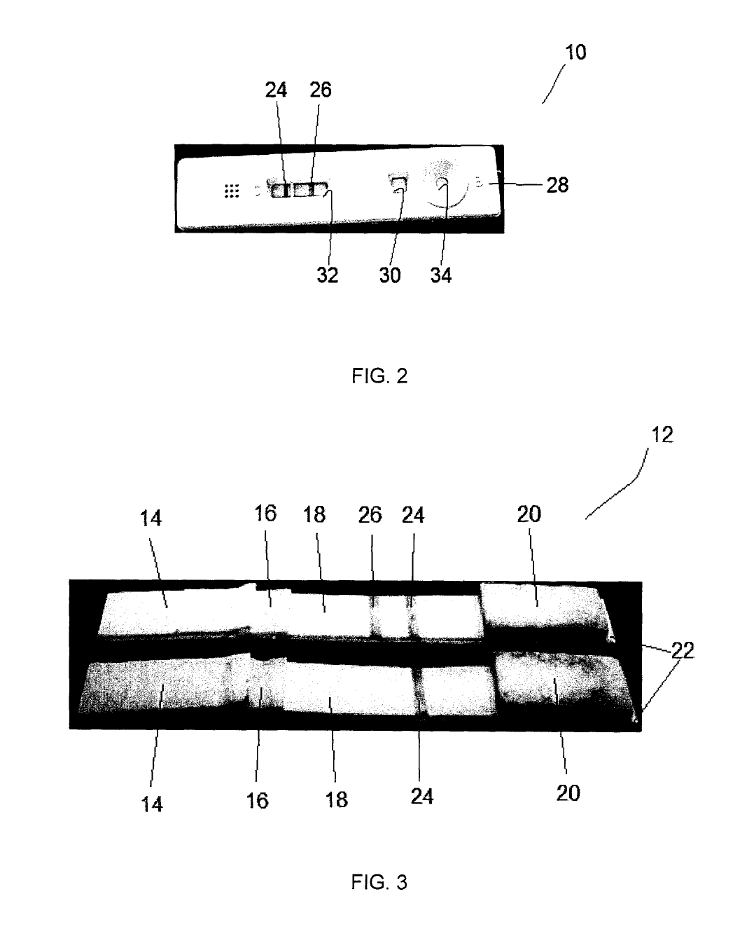

In one embodiment, the test strip (12) may be used directly or housed within a cassette (28) to facilitate handling. The cassette (28) comprises a housing and defines a sample aperture (30) for introducing the sample into the immunoassay (10), with the sample pad (14) positioned beneath the sample aperture (30) (FIGS. 1A and 1B). In one embodiment, the cassette (28) defines a buffer aperture (34) for introducing dilution buffer into the immunoassay (10) (FIG. 2). The cassette (28) comprises a test window (32) positioned above the control and test bands (24, 26) of the detection membrane (18), to permit visualization or detection of the control and test bands. The test window (32) is preferably formed of a transparent polymer material. Test results may thus be viewed through the test window (32) by eye, a detector, or reader system. Non-limiting examples of such devices include spectrophotometers, reflectance readers, luminometers, fluorometers, photodetectors or photomultiplier tubes, scintillation counters, and other suitable instruments.

The immunoassay (10) may be in the form of a kit which includes necessary antibodies, antigens, or buffered diluents as separate reagents, or in the form of a cassette (28) comprising all needed antibodies and antigens. A sample collector such as a finger prick needle may be included.

The immunoassay (10) is a qualitative test, providing a “yes” or “no” result in the form of a detectable signal, such as a color change or difference on the test strip. In one embodiment, the immunoassay (10) of the present invention comprises a competitive inhibition binding test. In such a test, a molecule competes with another molecule for binding to the same target In one embodiment, if the sample contains a sufficient amount of the analyte, a positive result is indicated by the absence of a detectable colored stripe at the test band (26). A negative result is indicated by the presence of a detectable colored stripe at the test band (26).

The threshold value which divides positive and negative results may be determined by the concentration or quantity of the labeled antibody and the first capture reagent. The immunoassay may be made more sensitive to lower concentrations of vitamin D by having a reduced quantity or concentration of the labeled antibody. Conversely, its sensitivity may be decreased by increasing the quantity or concentration of the labeled antibody, in which case a greater quantity of vitamin D will be required to block all the labeled antibody. In one embodiment, the immunoassay (10) is capable of identifying vitamin D deficiency where vitamin D levels are lower than about 32 ng/ml (80 nmol/L).

In one embodiment, the strength or intensity of the detectable signal may vary along a scale or gradient, and be related to the concentration of analyte in the sample. In this case, the immunoassay may be quantitative rather than qualitative.

In one embodiment, a detectable “control” signal forms at the control band (24) irrespective of the result at the test band (26). The control band indicates that the sample has flowed through the test strip (12) and that the test is valid and functioning properly. The control band is preferably downstream from the test band.

When the test strip is used, a fluid sample is added to the sample pad (14) for example, by pipette or medicine dropper. In one embodiment, the amount of the fluid sample ranges from about 1 μL to about 100 μL. In one embodiment, the amount of the fluid sample is about 20 μL. The sample may be pre-treated to facilitate the release of vitamin D from its binding protein or other agents in the sample. In one embodiment, the sample is treated with urea, guanidine hydrochloride, an acidic solution, or an alkaline solution. The sample migrates via capillary action from the sample pad (14) to the conjugate pad (16). The labeled antibody is mobilized by the movement of fluid through the conjugate pad (16), and is carried by the flow through the test strip (12). Dilution buffer may be added to provide sufficient testing volume, and to ensure optimum lateral capillary flow as the sample migrates through the entire length of the test strip (12). In one embodiment, dilution buffer is added separately from the sample, and may be added upstream from the sample pad.

If present in sufficient quantity, the vitamin within the sample interacts with the labeled antibody to form a complex, leaving no unbound labeled antibody (or an insignificant amount). The complex then migrates from the conjugate pad (16) to the detection membrane (18). The labeled antibody, which is already bound to the analyte, subsequently does not bind to the capture reagent immobilized at the test band (26). The absence of a detectable colored stripe at the location of the test band (26) indicates the presence of the analyte in the sample (i.e., a positive result; FIG. 1A; FIG. 3, lower panel).

If there is insufficient vitamin to bind all or substantially all of the labeled antibody, the unbound labeled antibody will form a detectable signal in the test band. In one embodiment, the labeled antibody comprises colloidal gold which forms a colored stripe visible to the naked eye. This negative result indicates vitamin D deficiency in the sample (FIG. 1B; FIG. 3, top panel). In one embodiment, the detectable signal is observed in about ten minutes or less.

The sample and dilution buffer liquid moves past the control and test bands (24, 26) and collects in the wick (20) which prevents the backflow of the fluid into the immunoassay (10) or accidental leakage of the fluid following testing.

Methods for preparing and isolating monoclonal antibodies are well known in the art. See, for example, U.S. Pat. No. 7,776,544 to Gupta; Coligan et al. (1995); Sambrook et al. (2001); and Hurrell (1982), the contents of which are incorporated herein by reference, where permitted.

As used herein, the term “subject” means humans or animals. It will be appreciated by those skilled in the art that the method of the present invention has diagnostic and therapeutic applications, including screening for vitamin D deficiency; monitoring the effects of treatment to alleviate the deficiency; and assessing a patient's susceptibility to chronic diseases including, but not limited to, high blood pressure, tuberculosis, cancer, periodontal disease, multiple sclerosis, chronic pain, depression, Parkinson's and Alzheimer's disease, schizophrenia, seasonal affective disorder, peripheral artery disease, and several autoimmune diseases including rheumatoid arthritis and type 1 diabetes.

The immunoassay (10) is rapid, accurate and inexpensive due to the reduced volume of sample and reagents (i.e., microlitres), inexpensive and disposable materials, and minimal testing steps. A sample can be easily collected by finger tip puncture. Fluid flow manipulation is governed by capillary action through the test strip. Fluid handling, separation and detection functionalities are conveniently integrated within the immunoassay (10). Samples may thus be processed rapidly in minutes, compared to current time-consuming technologies, for example, liquid chromatography-tandem mass spectrometry, radio-immunoassays, solvent extraction and HPLC, which require a high degree of proficiency, extensive training and expensive equipment. The immunoassay (10) thus reduces costs for both the patient and healthcare system since the results may be obtained within minutes of performing the test, either at home or at a point-of-care location. In one embodiment, the immunoassay (10) takes approximately ten minutes, preferably less than ten minutes.

Exemplary embodiments of the present invention are described in the following Examples, which are set forth to aid in the understanding of the invention, and should not be construed to limit in any way the scope of the invention as defined in the claims which follow thereafter.

### Example 1

### Detection of Vitamin D in a Fluid Sample

A lateral flow immunoassay was used to detect vitamin D₃ in a fluid sample. Within the test strip, the detection membrane comprised nitrocellulose membrane immobilized with 25-hydroxy vitamin D₃ conjugated to bovine serum albumin (BSA) at the test band. Anti-mouse IgG antibodies were immobilized at the control band. The particulate conjugate comprised monoclonal antibodies against 25-hydroxy vitamin D₃ labelled with colloidal gold (40 nm).

Anti-vitamin D antibody was raised in mice by immunizing animals with purified antigen conjugated to keyhole limpet hemocyanin. Different clones were obtained after fusion and their activity was checked against 25-hydroxy vitamin D₃. A clone specific for 25-hydroxy vitamin D₃ was selected and further re-cloning was performed. The clone that showed the highest titer in the ELISA assay using 25-hydroxy vitamin D₃ coated plate was selected. Cells were grown in cell culture medium and supernatant rich in antibody was collected. The antibody rich cell supernatant was purified using a Protein G™ column (GE Healthcare Life Sciences, Baie d′Urfe, Quebec, Canada). An isotyping kit was used to identify the antibody class. The antibody was confirmed as IgG1 and [symbol] side chain.

A fluid sample was prepared which contained BSA-vitamin D₃ conjugate (approximately 40 ng/ml of vitamin D₃). 20 μl of the test sample was applied to the sample pad. 160 μl (7 drops) of dilution buffer was added to facilitate the migration of the test sample across the strip. No color formation was observed on the test band of the detection membrane (FIG. 3, lower panel shows a stripe only in the control position). This result indicates that vitamin D₃ present in the fluid sample bound to the colloidal gold-labelled antibody, thereby blocking it from binding to 25-hydroxy vitamin D₃-BSA immobilized at the test band.

20 μl of 1 mg/ml BSA in phosphate buffered saline was applied to the sample pad and chased with 160 μl (7 drops) of dilution buffer. Color formation was observed on the test band (FIG. 3, top panel shows a stripe in both the test and control positions). This result indicates that the since the fluid sample lacked vitamin D₃, the colloidal gold-labelled antibody was able to bind to 25-hydroxy vitamin D₃-BSA immobilized at the test band.

### Example 2

### Assessment of the Analytical Performance of the Immunoassay

The immunoassay was evaluated by using 25-hydroxy vitamin D₃ conjugated to bovine serum albumin (D-BSA) of known vitamin D concentration. 20 μL of known concentrations of D-BSA were applied on the test strip followed by 7 drops of chase buffer and the appearance of test and control lines was checked after 10 minutes. The assay was repeated to confirm the reproducibility of the results (Table 1).

TABLE 1
Test Results

| | 1st Set | | | 2nd Set | | |
|---|---|---|---|---|---|---|
| D-BSA Sample | Test | No Test | Control | Test | No Test | Control |
| (ng/ml) | Line | Line | Line | Line | Line | Line |
| 188 | | ✓ | ✓ | | ✓ | ✓ |
| 125 | | ✓ | ✓ | | ✓ | ✓ |
| 62 | | ✓ | ✓ | | ✓ | ✓ |
| 4.7 | ✓ | | ✓ | ✓ | | ✓ |
| 0 | ✓ | | ✓ | ✓ | | ✓ |

The test line appeared up to a vitamin D concentration of 4.7 ng/ml. There were no test lines at vitamin D concentrations of 62 ng/ml or greater. The results were consistent in both assays. The control line appeared in all the test strips, confirming the validity and proper performance of the assays. These results demonstrate that variations in vitamin D concentrations and the corresponding presence or absence of the test line provide useful information of vitamin D deficiency or sufficiency, respectively.

A reference serum sample containing 15 ng/ml of vitamin D was spiked with various known concentrations of vitamin D. 20 μL of serum samples with or without spiking were applied to the test strip followed by 7 drops of chase buffer. The appearance of test and control lines was checked after 10 minutes. Two different lots of the test strip were used to confirm the findings. The results are shown in Table 2.

TABLE 2
Laboratory Evaluation of Immunoassay

| Serum Vitamin D | Vitamin D | Final Vitamin D | | No | |
|---|---|---|---|---|---|
| concentration | spike | concentration | Test | Test | Control |
| (ng/ml) | (ng/ml) | (ng/ml) | Line | Line | Line |
| 15 | 0 | 15 | ✓ | | ✓ |
| 15 | 0 | 15 | ✓ | | ✓ |
| 15 | 25 | 40 | | ✓ | ✓ |
| 15 | 50 | 65 | | ✓ | ✓ |
| 15 | 50 | 65 | | ✓ | ✓ |
| 15 | 100 | 115 | | ✓ | ✓ |
| 15 | 100 | 115 | | ✓ | ✓ |
| 15 | 150 | 165 | | ✓ | ✓ |
| 15 | 200 | 215 | | ✓ | ✓ |

The results indicate that the test line appeared only at 15 ng/ml vitamin D concentration. There were no test lines at vitamin D concentrations of 40 ng/ml and higher concentrations. The results were consistent with different lots of vitamin D. The control line appeared in all the test strips, confirming the validity and proper performance of the assays.

### Example 3

### Comparison of immunoassay to conventional assay

The immunoassay of the present invention yields qualitative results which are consistent with results obtained using a conventional quantitative 25-hydroxy vitamin D₃ assay (LIAISON™ 25 OH Vitamin D TOTAL Assay, DiaSorin Canada Inc., Mississauga, ON, Canada). Table 3 compares the results obtained for the same samples using the two different assays.

TABLE 3
Comparison of immunoassay to conventional vitamin D test

| | Quantitative | | | |
|---|---|---|---|---|
| | Test results | | | |
| | (Liaison ™, | | Qualitative Test results of | |
| | DiaSorin) | | immunoassay | |
| | 25(OH) | | test strip (Yes/No) | |
| Patient # | Vit D nmol/L | Result | Strip Result | Strip Comment |
| 1 | 125 | Sufficient | Sufficient | No Test Line |
| 2 | 113 | Sufficient | Sufficient | No Test Line |
| 8 | 92 | Sufficient | Sufficient | No Test Line |
| 9 | 80 | Sufficient | Sufficient | No Test Line |
| 10 | 74 | Deficient | Deficient | Test Line |
| 11 | 58 | Deficient | Deficient | Test Line |
| 12 | 35 | Deficient | Deficient | Test Line |

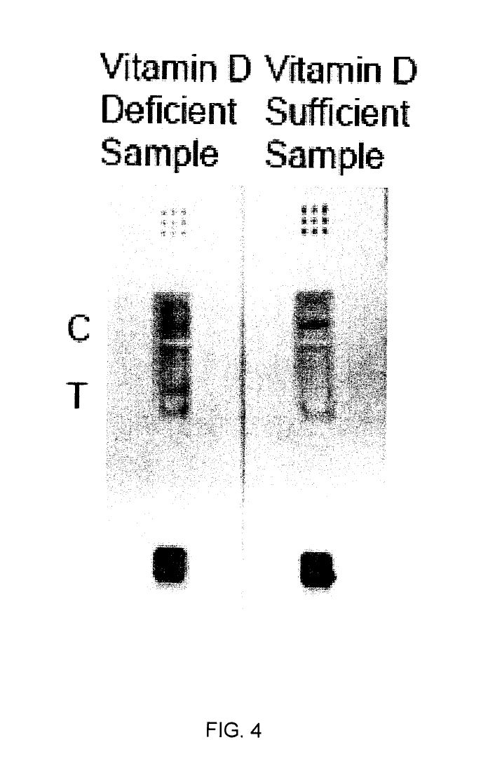

FIG. 4 shows actual test results from the immunoassay of the present invention comparing vitamin D deficient and sufficient blood samples. In the vitamin D deficient sample, both the control (C) and test (T) lines appear. In the vitamin D sufficient sample, only the control line (C) appears. In addition, it was observed that sufficient vitamin D sample made the test line disappear.

### REFERENCES

The following references are incorporated herein by reference (where permitted) as if reproduced in their entirety. All references are indicative of the level of skill of those skilled in the art to which this invention pertains.
Binkley, N., Krueger, D., Cowgill, C. S., Plum, L., Lake, E., Hansen, K. E., DeLuca, H. F. and Drezner, M. K. (2004) Assay variation confounds the diagnosis of hypovitaminosis D: a call for standardization. J Clin Endocrinol Metab 89(7):3152-57.
Carter, G. D. (2009) 25-hydroxyvitamin D assays: the quest for accuracy. Clin Chem 55(7):1300-02.
Clemens, T. L., Adams, J. S., Nolan, J. M. and Holick, M. F. (1982) Measurement of circulating vitamin D in man. Clin Chim Acta 121(3):301-8.
Coligan, J. E., Kruisbeek, A. M., Margulies, D. H., Shevach, E. M. and Strober, W. (Eds.) (1995) Current Protocols in Immunology. National Institutes of Health, John Wiley and Sons, Inc.
Gupta, R. Extraction and quantification of vitamins A & D in fluid samples. U.S. Pat. No. 7,776,544, issued Aug. 17, 2010.
Hurrell, J. G. R. (Ed.) (1982) Monoclonal Hybridoma Antibodies: Techniques and Applications. CRC Press, Inc., Boca Raton, Fla.
Hollis, B. W. (2004) Editorial: The determination of circulating 25-hydroxyvitamin D: no easy task. J. Clin Endocrinol Metab 89(7):3149-51.
Jones, K. D. and Cox, D. Lateral flow format, materials and methods. U.S. Pat. No. 7,867,780, issued Jan. 11, 2011.
Lawler, M., Pritchard, D., Black, M. and Orning, L. An assay for detecting vitamin D and antibodies therefor. International Publication No. WO 2010/100487 A1, published Sep. 10, 2010.
Manabe, T. Chromatographic test device. United States Patent Application Publication No. 2009/0246861 A1, published Oct. 1, 2009.
Quesada, J. M., Mata-Granados, J. M. and Luque de Castro, M. D. (May 2004) Automated method for the determination of fat-soluble vitamins in serum. J Steroid Biochem Mol Biol. 89-90(1-5):473-7. Proceedings of the 12^{th} Workshop of Vitamin D.
Sambrook, J. and Russell, D. (2001) Molecular Cloning: A Laboratory Manual. Third Edition. Cold Spring Harbor Laboratory Press, Cold Spring Harbor, N.Y.
Snellman, G., Melhus, H., Gedeborg, R., Byberg, l., Berglund, L., Wernoth, L. and Michaelsson, K. (2010) Determining vitamin D status: a comparison between commercially available assays. PLoS ONE 5(7):e11555.
Wagner, D., Hanwell, H. E. C. and Vieth, R. (2009) An evaluation of automated methods for measurement of serum 25-hydroxyvitamin D. Clin Biochem. 42:1549-1556.
Wild, D. (Ed.) (2005) The Immunoassay Handbook, 3^{rd} Ed. Elsevier Ltd., Oxford, UK.
Zerwekh, J. E. (2004) The measurement of Vitamin D: analytical aspects. Ann Clin Biochem. 41(Pt 4):272-281.
Zweig, S. E. Fluorescence lateral flow immunoassay. United States Patent Application Publication No. 2006/0263907 A1, published Nov. 23, 2006.

1. A method of screening a fluid sample for a threshold value of vitamin D, comprising the steps of:
(a) obtaining a fluid sample from a subject;
(b) applying the sample to a lateral flow test strip comprising a conjugate pad comprising a known quantity of a labeled antibody against vitamin D;
(c) allowing an immunocomplex to form between the labeled antibody and any vitamin D in the sample;
(d) flowing the labeled antibody or immunocomplex through a test band comprising an immobilized first capture reagent capable of binding to:
i. the labeled antibody but not the immunocomplex, or
ii. the immunocomplex but not the labeled antibody; and
(e) determining whether or not the amount of vitamin D in the sample exceeds a threshold value by detecting or not detecting the labeled antibody in the test band.

2. The method of claim 1, wherein before step (b), the sample is treated to facilitate the release of vitamin D from a binding protein or a binding agent.

3. The method of claim 2, wherein the sample is treated with urea, guanidine hydrochloride, an acidic solution, or an alkaline solution.

4. The method of claim 1, wherein the sample is a body fluid selected from urine, blood, plasma, serum, saliva, ocular fluid, spinal fluid, or perspiration.

5. The method of claim 4, wherein the sample is a body fluid selected from serum or blood.

6. The method of claim 1, wherein the labeled antibody is anti-25-hydroxy vitamin D₃, anti-25-hydroxy vitamin D₂, or anti-1,25-dihydroxy vitamin D₃.

7. The method of 1, wherein the antibody is labeled with a label selected from a chromogen, a catalyst, a fluorescent compound, a chemiluminescent compound, a colloidal gold, a dye particle, or a latex particle tagged with a detector reagent.

8. The method of 7, wherein the label comprises colloidal gold.

9. The method of claim 1, wherein the first capture reagent comprises 25-hydroxy vitamin D₃, 25-hydroxy vitamin D₂, or 1,25-dihydroxy vitamin D₃.

10. The method of claim 1, wherein the first capture reagent comprises an antibody specific to vitamin D3.

11. The method of claim 9, wherein the first capture reagent is conjugated to a bulky protein, which comprises bovine serum albumin, keyhole limpet hemocyanin, thryoglobulin, or ovalbumin.

12. The method of claim 1, wherein the detection membrane further comprises a control band comprising a second capture reagent which binds to either the labeled antibody or the immunocomplex.

13. The method of claim 12, wherein the second capture reagent comprises an antibody specific to the labeled antibody.

14. The method of claim 1, wherein the lateral flow test strip is housed within a cassette defining a sample aperture for introducing the sample and a test window for viewing a test result.

15. The method of claim 14, wherein the cassette defines a buffer aperture for introducing dilution buffer.

16. The method of claim 1, wherein the amount of the sample ranges from about 1 μL to about 100 μL.

17. The method of claim 1, wherein the detectable signal is observed in about ten minutes or less.

18. A lateral flow immunoassay comprising a lateral flow test strip comprising a conjugate pad capable of releasing a labelled antibody against vitamin D, whereby the labeled antibody and any sample vitamin D forms an immunocomplex, and a detection membrane comprising an immobilized capture reagent capable of binding to i. the labeled antibody but not the immunocomplex, or ii. the immunocomplex but not the labeled antibody.

19. A kit comprising the lateral flow test strip of claim 18, a sample collector, and a diluent.
